(19) United States
(12) Patent Application Publication
(10) Pub. No.: US 2005/0037077 A1
(43) Pub. Date: Feb. 17, 2005

(54) Galenic microparticulate oral formulation for delayed and controlled release of pharmaceutical active principles

(76) Inventors: Valerie Legrand, Lyon (FR); Catherine Castan, Orlienas (FR); Remi Meyrueix, Lyon (FR); Gerard Soula, Meysieu (FR)

Correspondence Address: FINNEGAN, HENDERSON, FARABOW, GARRETT & DUNNER;LLP, 1300 I STREET, NW, WASHINGTON, DC, 20005, US

(21) Appl. No.: 10/492,129

(22) PCT Filed: Oct. 9, 2002
(86) PCT No.: PCT/FR02/03443

(30) Foreign Application Priority Data
Oct. 9, 2001 (FR) 01/12999

(51) Int. Cl. A61K 9/26

(52) U.S. Cl. 424/469

(57) ABSTRACT

The invention relates to a microparticulate system for the delayed and controlled release of active principles (AP) whose absorption window in vivo is essentially limited to the upper parts of the gastrointestinal tract, this system being intended for oral administration. The object of the invention is to provide a system ensuring that the AP is released with certainty by means of a dual mechanism of “time-dependent” and “pH-dependent” release. To achieve this object, the invention proposes a multimicrocapsular oral galenical form which is designed so as to guarantee therapeutic efficacy, and in which the release of the AP is governed by a dual release triggering mechanism that is “time-triggering” and “pH-triggering”. This system consists of microcapsules (200 to 600 μm) comprising a core of AP coated with a film (maximum 40% by weight) comprising a hydrophilic polymer A (Eudragit® L) and a hydrophobic compound B (vegetable wax, melting point=40-90° C.), B/A being between 0.2 and 1.5. These microcapsules have a dissolution behavior in vitro such that, at a constant pH of 1.4, a latency phase of between 1 and 5 hours is observed, followed by a release of the AP, and such that the change from pH 1.4 to pH 6.8 results in a release of the AP without a latency period in vitro.

The present invention relates to the field of microparticulate systems for the delayed and controlled release of one or more active principles, AP, intended for oral administration.

The AP envisaged in the present invention are those whose absorption is essentially limited to the upper parts of the gastrointestinal tract located upstream from the colon (upstream from the ileocecal junction), and which represent a large majority of pharmaceutical active principles.

More precisely, the invention relates to a microparticulate galenical form for delayed and controlled release where the controlled release phase is triggered with certainty by means of a dual mechanism: “time-dependent” release triggered after a certain residence time in the stomach, and “pH-dependent” release triggered by a change in pH when the particles enter the small intestine, which starts without a latency period. The microparticles of the present invention are microcapsules containing at least one active principle (AP)—excluding perindopril—having a particle size of between 100 and 1200 microns and individually coated with a film allowing the delayed and controlled release of the AP.

Systems for the delayed and controlled release of AP are particularly useful in cases where it is desirable, for reasons of chronobiology, that the AP be “bioabsorbed” at a precise time of day so as to be in phase with the circadian cycle. This approach is appropriate to the treatment of cancer or hypertension, the administration of antiinflammatory drugs or the regulation of glycemia in the treatment of diabetes. It may be advantageous, for example, for the AP to be bioabsorbed very early in the morning so as to assure therapeutic cover when the patient wakes up, without compelling him to wake up prematurely. To do this, the galenical system ingested by the patient, for example after the evening meal, must allow a delayed release of the AP.

However, the first rule imposed on the pharmacist is to guarantee that the prescribed drug will be absorbed by the patient. In the case of a delayed release form, it is therefore crucial to have a total guarantee of release of the active principle at a given moment in order to obtain the therapeutic effect. Now, one is obliged to note that delayed release forms cannot ensure with certainty that the AP will be released after a prescribed time. This problem becomes particularly acute in the case where it is vital for the patient that this release does indeed take place, for example in the treatment of cardiovascular diseases or diabetes.

In fact, delayed release forms are conventionally obtained by coating the AP with a layer of enteric polymer, for example the methacrylic acid/methyl methacrylate copolymer EUDRAGIT® L. This type of enteric coating is known to have a reduced permeability under the acidic pH conditions of the stomach, and to dissolve when the pH increases to a value close to that prevailing in the small intestine, thereby releasing the AP. However, the intraindividual and interindividual variability of the gastric pH conditions and the gastric emptying time do not make it possible to ensure with certainty that the AP will be released after a given time.

Purely “time-dependent” delayed release systems, i.e. those for which the release of the AP is triggered after a given residence time in the gastrointestinal tract, are not satisfactory either. In fact, because of the intraindividual and interindividual variability of the gastric residence time, the AP may be released after it has passed its absorption window, which, for the majority of AP, is located in the upper part of the gastrointestinal tract. The bioabsorption may thus be very low or even zero.

In this context it would be particularly advantageous to have a galenical form for the delayed and controlled release of the AP which made it possible to assure with certainty the release of the AP by means of a dual AP release triggering mechanism: “time-dependent” release triggered after a controlled time in the stomach, without a change in pH, and “pH-dependent” release triggered by an increase in the pH when the galenical form enters the intestine. These two AP release triggering factors, in succession, would give the galenical system a high degree of reliability in use. The release of the AP would thus be guaranteed after a preset latency period, even if the variation in pH did not intervene as a trigger, i.e. even if the galenical form did not pass from the stomach into the intestine.

To minimize the interindividual variability of AP absorption, it is necessary to adjust the latency period preceding the release of the AP into the stomach by considering the physiological conditions of the gastrointestinal tract in man. According to the well-known results of Davis et al., J. of Controlled Release, 2, 27-38 (1985), the gastric residence time of a preparation is very variable, being in the order of 0.5 to 10 hours. It would therefore be particularly advantageous to have a galenical form which released the active principle into the stomach after a given constant latency period within this interval of 0.5-10 hours, so that the action time of the drug would be the same from one individual to another or even from one day to the next for the same individual.

Moreover, to optimize the bioavailability of AP whose absorption is mainly limited to the upper parts of the gastrointestinal tract, it would be advantageous if the “pH-dependent” release into the intestine were to take place without a latency period, since otherwise the AP would not be released in its absorption window and, consequently, the patient would not be treated.

Another unique advantage of such a system would be that, by mixing it with a galenical form for immediate release of the AP, or by mixing it with another galenical form for delayed and controlled release of the AP, it would afford release profiles which exhibited several AP release waves (one AP or several identical or different AP) or which, by appropriate adjustment of the different fractions, assured a constant plasma AP concentration level.

It would also be advantageous for the delayed and controlled release form to consist of a plurality of microcapsules with a diameter below 2000 microns. In fact, for such a form, the dose of AP to be administered is spread over a large number of microcapsules (typically 10,000 for a dose of 500 mg) and thus has the following intrinsic advantages:
The residence time of the microcapsules in the upper parts of the gastrointestinal tract can be prolonged, thereby increasing the duration of passage of the AP through the absorption windows and thus maximizing the bioavailability of the AP.
The use of a mixture of microcapsules with different delayed and controlled release profiles makes it possible to create release profiles which exhibit several release waves or which, by appropriate adjustment of the different fractions, assure a constant plasma AP concentration level.
The sensitivity to the variability of gastric emptying is reduced because the emptying, which in this case takes place over a large number of particles, is statistically more reproducible.
Bringing the tissues into contact with a high dose of AP—dose dumping—is avoided. Each microcapsule actually contains only a very small dose of AP. This eliminates the risk of tissue damage due to a local excess concentration of aggressive AP.
It is possible to combine several galenical forms (immediate and/or delayed and/or prolonged release), containing one or more active principles, in these “multimicrocapsular” systems.
It is possible to present these microcapsules in the form of sachets, gelatin capsules or tablets. In cases where the dose of AP is high (500 mg or more), the monolithic forms are too large to be swallowed easily. It is then of particular value to have a microparticulate form for delayed and controlled release of the AP, which those skilled in the art can formulate as disintegrating tablets or sachets.

Finally, it would also be desirable for the coating film around the microcapsules to be thin. In fact, a thick coating would have several adverse consequences:
(a) the mass fraction of excipient in the galenical form would be too high, making the mass of the drug too large to be swallowed easily and hence, in fine, creating compliance problems that jeopardize the success of the treatment; and
(b) the microcapsules would take a very long time to manufacture.

In summary, it would therefore be of particular value to have a microparticulate oral galenical form for the delayed and controlled release of AP which simultaneously possessed the following properties:
the release of the AP can be triggered in two ways:
by release dependent on time, also called “time-dependent” release, when the residence time of the particles in the stomach exceeds 5 hours;
by release dependent on a variation in pH, also called “pH-dependent” release, which starts without a latency period when the system enters the intestine and the pH increases; these two AP release triggering factors, in succession, guarantee that the AP is released after a preset latency period, even if the variation in pH has not intervened as a trigger;
it consists of a plurality of small microcapsules of coated AP; and
the mass fraction of coating excipients is limited.

The delayed or controlled release of AP has formed the subject of numerous studies.

Thus PCT patent application WO-A-96/11675 describes microcapsules for the oral administration of medicinal and/or nutritional active principles (AP) whose size is less than or equal to 1000 μm. These microcapsules consist of particles coated with a coating material consisting of a mixture of a film-forming polymeric derivative (ethyl cellulose), a hydrophobic plasticizer (castor oil), a surfactant and/or lubricant (magnesium stearate) and a nitrogen-containing polymer (polyvinylpyrrolidone: PVP). These microcapsules are also characterized by their ability to reside for a long time (at least 5 hours) in the small intestine and, during this residence time, to allow absorption of the AP over a period greater than the natural transit time in the small intestine.

The microcapsules according to said patent application do not provide a solution to the particular problem of the delayed and controlled release of AP with a “time-dependent” and “pH-dependent” triggering of the AP.

Patent application FR-A-00 14876 describes a drug for the treatment of type II diabetes which comprises several thousand antihyperglycemic microcapsules (metformin) each consisting of a core containing at least one antihyperglycemic, and of a coating film (e.g. stearic acid and ethyl cellulose) applied to the core, which allows prolonged release of the antihyperglycemic in vivo. These microcapsules have a particle size of between 50 and 1000 μm.

Said patent application FR-A-00 14876 does not indicate how to obtain the delayed and controlled release of AP with a “time-dependent” and “pH-dependent” triggering of the AP.

European patent application EP-A-0 609 961 discloses oral morphine granules for which the controlled release of the AP accelerates with the increase in pH.

These granules consist of:
a sugar core (φ=100 to 1700 μm)
coated with a layer of active ingredient associated with a binder (PVP or hydroxypropyl methyl cellulose: HPMC),
and an outer envelope based on:
   a polymer that is insoluble independently of the pH (ethyl cellulose or methacrylic acid ester/ammonium methacrylate copolymer: EUDRAGIT® RS or RL),
   an enteric polymer that is insoluble at acidic pH (methacrylic acid/methyl methacrylate copolymer: EUDRAGIT® L),
   a component that is partially soluble at acidic pH (polyethylene glycol, PVP, HPMC, polyvinyl alcohol: PVA),
   optionally a plasticizer (diethyl phthalate)
   and optionally a filler (talcum).

The mass fractions of AP are e.g. 41%, 38% and 29% and the mass fractions of outer envelope are e.g. 14.1%, 21.5% and 12.3% (by weight).

Release of the AP takes place at any pH and increases as the pH changes from 1.2 to 7.5. This is therefore a form for prolonged and non-delayed release.

The article by H. YOSHINO entitled “Design and evaluation of time-controlled release systems for site-specific oral drug delivery to the GI tract”, published in Current status on targeted drug delivery to the GI tract, Capsugel library, Symp. Ser., Short Hills 22/04, London 6/05, Tokyo 14/05, pp 185-190, (1993), describes multiparticulate oral galenical systems for delayed and controlled release induced by an organic acid and by the residence time in the GIT. These systems are made up of 1000 μm microcapsules consisting of a neutral sugar core coated with a layer of active ingredient mixed with an organic acid (succinic acid), and of an outer layer of methacrylic acid ester/ammonium methacrylate copolymer (EUDRAGIT® RS). The organic acid is described as allowing a rapid release of the AP after the latency phase. This organic acid is transported by the water which has entered the microcapsules through the enteric outer layer. It then works towards modifying the permeability of the coating to allow rapid diffusion of the AP out of the microcapsules. The presence of this acid in intimate contact with the AP can be detrimental to the latter.

U.S. Pat. No. 6,033,687 describes a formulation consisting of a mixture of two types of granules (φ=1.4 mm) based on diltiazem, namely granules with a short latency period and granules with a long latency period. The release profiles are measured at pH 1. These granules comprise:
a neutral sugar core (φ=0.5-1.5 mm),
a layer of diltiazem associated with a binder (hydroxypropyl cellulose, carboxymethyl cellulose, ethyl cellulose, polyvinylpyrrolidone, alginate, EUDRAGIT),
and a single outer coating based on a lubricant (talcum), two methacrylic acid ester/ammonium methacrylate copolymers (EUDRAGIT® RS and EUDRAGIT® RL), a surfactant (sodium laurylsulfate) and a plasticizer (triethyl citrate).

In the granules with a short latency period, the mass fraction of the coating represents 12.3%, compared with 30.3% in the granules with a long latency period. However, this technique does not afford long latency periods for film coating rates below 30%. Furthermore, in view of the intraindividual and interindividual variability of the gastric residence time, this “time-dependent” delayed release system may release the AP after it has passed its absorption window. This results in a substantial loss of bioavailability.

Patent EP-B-0 263 083 describes a microcapsule coating composition that affords a zero-order and reproducible AP release profile. This coating composition is composed of a mixture of:
a hardening polymer to assure the mechanical strength of the coating, possible examples being ethyl cellulose or methacrylic acid copolymer(s) (EUDRAGIT® E, L, S or RS),
a lipophilic compound, e.g. stearic acid or paraffin,
and talcum.

This coating composition is present in the microcapsules in an amount of e.g. 15 to 35% by weight. The hardening polymer/lipophilic compound ratios are e.g. 44 and 42% respectively in Examples 4 and 5.

The profiles obtained are profiles without a latency period of variable duration. Said patent neither teaches nor mentions how to obtain a profile with a delayed and controlled release that is triggered at the end of the latency period and/or by a variation in pH.

Patent application WO-A-01/58424 A1 discloses “floating” microcapsules coated with an enteric coating based e.g. on EUDRAGIT® L, magnesium stearate, talcum and a plasticizer such as dibutyl sebacate. This coating can be enveloped in a “bioadhesive” film based on chitosan. Like every enteric coating, the aim of the enteric coating according to patent document WO-A-01/58424 is a “pH-dependent” release rather than the conjunction of a “time-dependent” release and a “pH-dependent” release. Furthermore, FIGS. 1 to 3 of said patent application show that the simple objective of “pH-dependent” release is very imperfectly achieved since up to 20% of the AP is released in two hours only at constant acidic pH. As the particles described in said patent application float in the stomach, their gastric residence time is described as increased, so much so that one may even fear the absence of any “pH-triggered” release. Finally, the release would take place in an uncontrolled manner due to the rogue leaks of AP into the stomach.

European patent application EP-A-1 101 490 relates to a pharmaceutical preparation that is capable of releasing an active principle into the large intestine and more particularly the colon. This preparation can consist of tablets or granules comprising a core and a coating.

The technical problem underlying said invention is to propose a pharmaceutical form that is capable of allowing the release of a medicinal substance at a target site in the lower part of the small intestine, the ascending colon, the transverse colon or the lower part of the large intestine. Given the fact that the mean residence time in the stomach is 5 hours and that, on average, a further 2 hours are required to reach the lower part of the small intestine, the preparation according to EP-A-1 101 490 is designed so that the medicinal substance is not released for 5 hours under acidic conditions simulating the stomach, and is only released after a latency period of at least 2 hours in a fluid simulating the pH conditions of the intestine (cf. especially claim 7 of EP-A-1 101 490).

It is therefore apparent that this system aimed at medicinal substances absorbed in the lower parts of the intestine (colon) is not suitable for medicinal substances mainly absorbed in the upper parts of the gastrointestinal tract. Moreover, the system according to European patent application EP-A-1 101 490 does not make provision for release of the AP by means of a dual release triggering mechanism:
release into the stomach after a constant given latency period within an interval of 0.5-10 hours (“time-dependent” mechanism),
and release without a latency period after entering the intestine (“pH-dependent” mechanism).

Finally, the problem of the interindividual or intraindividual variability of the gastric residence time is not solved by the preparation according to EP-A-1 101 490.

Thus the prior art does not comprise a galenical system that makes it possible to delay and to guarantee with certainty the release of AP preferentially absorbed in the upper parts of the gastrointestinal tract, by means of a dual release mechanism:
“time-dependent” release after a latency period in the stomach which has the characteristic of being a constant given latency period within an interval of 0.5-10 hours,
and “pH-dependent” release without a latency period.

In view of this state of the art, one of the essential objectives of the present invention is to provide a novel multimicroparticulate galenical system for the oral administration of active principles essentially absorbed in the upper parts of the gastrointestinal tract, this system being of the delayed and controlled release type that assures the release of the AP with certainty and hence guarantees the therapeutic efficacy of said system, by means of a dual “time-dependent” and “pH-dependent” release mechanism. These two AP release triggering factors, in succession guarantee the release of the AP after a preset latency period, even if the variation in pH has not intervened as a trigger.

One essential objective of the present invention is to propose a galenical form made up of a plurality of microcapsules that makes it possible to escape from the interindividual and intraindividual variability of the gastric emptying time by releasing the AP at pH 1.4 according to a delayed release profile which has a latency period with an adjustable given duration of between 0.5 and 10 hours, followed by a release phase that starts without a latency period.

One essential objective of the present invention is to propose a galenical form made up of a plurality of microcapsules that makes it possible on the one hand to release the AP according to a delayed release profile at pH 1.4 with a constant given latency period of between 0.5 and 10 hours, and according to a release half-life t_{1/2} of between 0.25 and 35 hours, and on the other hand to release the AP when the pH changes from 1.4 to 6.8, without a latency period and with a t_{1/2} of between 0.25 and 20 hours.

One essential objective of the present invention is controlled when the pH changes from 1.4 to 6.8.

One objective of the present invention is to propose a galenical form consisting of a large number of microcapsules, for example in the order of several thousand, this multiplicity statistically assuring a good reproducibility of the AP transit kinetics throughout the gastrointestinal tract, the result being a better control of the bioavailability and hence a better efficacy.

One essential objective of the present invention is to propose a galenical form made up of a plurality of coated microcapsules that avoids the use of large amounts of coating agent, the mass fraction of coating agent being comparable to that of monolithic forms.

One essential objective of the present invention is to propose a pharmaceutical form made up of a plurality of coated microcapsules that makes it possible to present the AP in a form that is easy to swallow, namely a sachet or a disintegrating tablet.

One essential objective of the present invention is to propose a pharmaceutical form made up of a plurality of coated microcapsules that makes it possible to mix several different active principles.

Another objective of the present invention is to propose a pharmaceutical form made up of a plurality of coated microcapsules each containing a neutral core.

Having set themselves the above objectives, among others, it was to the inventors' credit to have developed, in order to assure a certain release of AP mainly absorbed in the upper parts of the gastrointestinal tract and a good bioabsorption of pharmaceutical active principles, a multimicrocapsular galenical system which:
guarantees the absorption of the AP in its absorption window, which is mainly limited to the upper parts of the gastrointestinal tract;
thereby assures a certain therapeutic efficacy of this system or of this galenical form;
and has the essential characteristic of a dual triggering of the AP release.

This represents a major advance compared with the AP controlled release systems known hitherto, in which the release of the AP is triggered by a single factor, namely the residence time in the gastrointestinal tract for some systems and a variation in pH for other systems.

Thus the invention, which satisfies the objectives laid out above, among others, relates to a microparticulate oral galenical form for the delayed and control release of at least one AP—excluding perindopril—this AP having an absorption window in vivo that is essentially limited to the upper parts of the gastrointestinal tract,

said form being designed so as to guarantee its therapeutic efficacy by guaranteeing its absorption in vivo, and being characterized in that:

the release of the AP is governed by two different triggering mechanisms, one being based on a variation in pH and the other allowing the release of the AP after a predetermined residence time in the stomach,
and its dissolution behavior in vitro (determined as indicated in the European Pharmacopeia, 3rd edition, under the title: “Dissolution test for solid oral forms”: type II dissolutest performed under SINK conditions, maintained at 37° C. and agitated at 100 rpm) is such that:
   at a constant pH of 1.4, the dissolution profile includes a latency phase with a duration less than or equal to 5 hours, preferably of between 1 and 5 hours,
   and the change from pH 1.4 to pH 6.8, during the latency phase, results in a release phase that starts without a latency period.

In one preferred embodiment of the invention, the microparticulate oral galenical form consists of a plurality of microcapsules containing at least one active principle (AP) mainly absorbed in the upper parts of the gastrointestinal tract—excluding perindopril—these microcapsules being of the type that:
consist of particles of AP each coated with at least one film, this coating film consisting of a composite material which:
   comprises:
      at least one hydrophilic polymer A carrying groups that are ionized at neutral pH,
      and at least one hydrophobic compound B;
   and represents a mass fraction (% by weight, based on the total mass of the microcapsules) of ≦40;
and have a diameter below 2000 microns, preferably of between 200 and 800 microns and particularly preferably of between 200 and 600 microns,
   characterized in that their coating film consists of a composite based on A and B in which:
the weight ratio B/A is between 0.2 and 1.5, preferably between 0.5 and 1,
and the hydrophobic compound B is selected from products that are crystalline in the solid state and have a melting point T_{fB} such that T_{fB}≧40° C., preferably T_{fB}≧50° C. and particularly preferably 40° C.≦T_{fB}≦90° C.

According to one preferred characteristic of the invention, the hydrophilic polymer A is selected from:
(meth)acrylic acid/alkyl (e.g. methyl) (meth)acrylate copolymers (EUDRAGIT® S or L) and mixtures thereof;
cellulose derivatives, preferably cellulose acetate and/or phthalate, hydroxypropyl methyl cellulose phthalate and hydroxypropyl methyl cellulose acetate and/or succinate;
and mixtures thereof.

The preferred polymers A are (meth)acrylic acid/alkyl (e.g. methyl) (meth)acrylate copolymers. These copolymers, which are e.g. of the type marketed by RÖHM PHARMA POLYMERS under the registered trade marks EUDRAGIT® L and S series (for example EUDRAGIT® L100, S100, L30D-55 and L100-55), are anionic enteric (co)polymers soluble in aqueous media at pH values above those encountered in the stomach.

According to another preferred characteristic of the invention, the compound B is selected from the following group of products:
vegetable waxes, taken on their own or in mixtures with one another, such as those marketed under the marks DYNASAN® P60 and DYNASAN® 116, inter alia;
hydrogenated vegetable oils, taken on their own or in a mixture with one another, preferably selected from the group comprising hydrogenated cottonseed oil, hydrogenated soybean oil, hydrogenated palm oil and mixtures thereof;
monoesters and/or diesters and/or triesters of glycerol with at least one fatty acid, preferably behenic acid, taken by themselves or in a mixture with one another;
and mixtures thereof.

The AP release triggering mechanism without a variation in pH, after a predetermined residence time in the stomach, results especially from control of the hydration rate of the microcapsules and/or the dissolution rate of one or more components of the microcapsules. For example, and without implying a limitation, the hydration of the microcapsule can be controlled by:
the presence, in the microcapsules, of hydrophilic products that make it possible to adjust the osmotic pressure or to cause a swelling of the microcapsules;
regulation of the water permeability of the coating film;
the creation of a microporosity in the coating film;
or even the hydration or dissolution of a compound in the coating film.

One of the decisive advantages of the multimicrocapsular galenical system according to the invention for the delayed and controlled release of AP is that it involves, in vivo, two factors that trigger the release of the AP into the gastrointestinal tract, namely:
the residence time in the stomach: “time-triggered” release,
and the variation in pH: “pH-triggered” release.

These two AP release triggering factors are successive, so they give the galenical system a high degree of reliability in use. The release of the AP is thus guaranteed after a preset latency period, even if the variation in pH has not intervened as a trigger. The problems of interindividual variability are thus overcome. The therapeutic efficacy of the drug comprising such a galenical system is assured by observing a predetermined chronobiology adapted to the intended therapeutic performance.

In addition, in the case of the AP considered in the present invention whose absorption window is limited to the upper parts of the gastrointestinal tract, it is particularly advantageous if the form for delayed and then controlled release consists of a plurality of microcapsules. In fact, for such a form, the dose of AP to be administered is spread over a large number of microcapsules (typically 10,000 for a dose of 500 mg) and thus has the following intrinsic advantages:
The residence time of the microcapsules in the upper parts of the gastrointestinal tract can be prolonged, thereby increasing the duration of passage of the AP through the absorption windows and thus maximizing the bioavailability of the AP.
The use of a mixture of microcapsules with different delayed and controlled release profiles makes it possible to create release profiles which exhibit several release waves or which, by appropriate adjustment of the different fractions, assure a constant plasma AP concentration level.
The variability of the gastric emptying is reduced because the emptying, which in this case takes place over a large number of particles, is statistically more reproducible.
Bringing the tissues into contact with a high dose of AP—dose dumping—is avoided. Each microcapsule actually contains only a very small dose of AP. This eliminates the risk of tissue damage due to a local excess concentration of aggressive AP.
It is possible to present these microcapsules in the form of sachets, gelatin capsules or tablets. In cases where the dose of AP is high (500 mg or more), the monolithic forms are too large to be swallowed easily. It is then of particular value to have a microparticulate form for delayed and controlled release of the AP, which those skilled in the art can formulate as disintegrating tablets or sachets.

The multimicrocapsular galenical system according to the invention makes it possible to assure with certainty a delayed and controlled release of the AP into the GIT by means of two triggers, and thus to escape the interindividual and intraindividual variability of the gastric emptying conditions, while at the same time being economically viable and easy to ingest (optimized compliance).

According to one particularly advantageous characteristic of the preferred embodiment, at a constant pH of 1.4, the controlled release phase following the latency phase is such that the release time for 50% by weight of the AP (t_{1/2}) is defined as follows (in hours):
0.25≦t_{1/2}≦35
preferably 0.5≦t_{1/2}≦20

In practice, the release phase of the in vitro AP release profile at a constant pH of 1.4 has an adjustable release half-life.

According to another valuable characteristic of the preferred embodiment, the release phase following the change from pH 1.4 to pH 6.8, which takes place without a latency period, is such that the release time for 50% of the AP (t_{1/2}) is defined as follows (in hours):
0.25≦t_{1/2}≦20
preferably 0.5≦t_{1/2}≦15

Preferably, the microcapsules according to the invention comprise a single composite coating film AB. This simplifies their preparation and limits the coating rate.

Preferably, the AP is deposited on a neutral core with a diameter of between 200 and 800 microns, preferably of between 200 and 600 microns.

Without implying a limitation, the hydrophilic neutral core can contain sucrose and/or dextrose and/or lactose, or it can consist of a cellulose microsphere.

Advantageously, the microcapsule coating can comprise, in addition to the essential constituents A and B, other conventional ingredients known to those skilled in the art, such as especially:
colorants;
plasticizers, for example dibutyl sebacate;
hydrophilic compounds, for example cellulose and derivatives thereof or polyvinylpyrrolidone and derivatives thereof;
and mixtures thereof.

Advantageously, the AP is deposited by the techniques known to those skilled in the art, for example the technique of spray coating in a fluidized air bed onto neutral cores with a diameter of between 200 and 800 microns, preferably of between 200 and 600 microns.

From the quantitative point of view, the monolayer of coating agent represents at most 40% and preferably at most 30% by weight of the microcapsules. Such a limited coating rate makes it possible to produce galenical units each containing a high dose of active principle without exceeding a prohibitive size as regards swallowing. This can only improve compliance with the treatment and hence its success.

In qualitative terms, the AP of the microcapsules according to the invention is essentially absorbable in the upper parts of the gastrointestinal tract and is advantageously selected from one of the following families of active substances: antiulcer agents, antidiabetics, anticoagulants, antithrombics, hypolipidemics, antiarrhythmics, vasodilators, antiangina agents, antihypertensives, vasoprotectors, fertility promoters, labor inducers and inhibitors, contraceptives, antibiotics, antifungals, antivirals, anticancer agents, antiinflammatories, analgesics, antiepileptics, antiparkinsonian agents, neuroleptics, hypnotics, anxiolytics, psychostimulants, antimigraine agents, antidepressants, antitussives, antihistamines and antiallergics.

Reference may also be made to the list of active principles given on pages 4 to 8 of patent application EP-A-0 609 961.

Preferably, the AP is selected from the following compounds: metformin, acetylsalicylic acid, amoxicillin, pentoxifyllin, prazosin, acyclovir, nifedipine, diltiazem, naproxen, ibuprofen, flurbiprofen, ketoprofen, fenoprofen, indomethacin, diclofenac, fentiazac, estradiol valerate, metoprolol, sulpiride, captopril, cimetidine, zidovudine, nicardipine, terfenadine, atenolol, salbutamol, carbamazepine, ranitidine, enalapril, simvastatin, fluoxetine, alprazolam, famotidine, ganciclovir, famciclovir, spironolactone, 5-asa, quinidine, morphine, pentazocine, paracetamol, omeprazole, metoclopramide and mixtures thereof.

The microparticulate oral galenical form according to the invention can be a tablet, advantageously a tablet that disperses in the mouth, a powder or a gelatin capsule.

The microcapsules described above can be used for the manufacture of novel pharmaceutical or dietetic preparations of various AP which have optimized therapeutic or dietetic performance characteristics and are preferably presented in the form of tablets, advantageously disintegrating tablets and even more preferably tablets that disperse in the mouth, powders or gelatin capsules.

These microcapsules are all the more valuable because they are also perfectly tolerated by the organism, especially by the stomach, and furthermore can be obtained easily and economically.

The present invention further relates to these novel pharmaceutical or dietetic preparations as such, which are original in their structure, to their presentation and to their composition. Such pharmaceutical or dietetic preparations are administered orally, preferably as single daily doses.

It is pointed out that it may be of value to mix, in one and the same gelatin capsule, tablet or powder, at least two types of microcapsule whose release kinetics are different but within the framework characteristic of the invention.

It is also possible to mix the microcapsules according to the invention with a certain amount of AP that is immediately available in the organism.

It can also be envisaged to associate microcapsules containing different AP.

In addition, a further subject of the invention is a galenical (pharmaceutical or dietetic) system, preferably in the form of a tablet, advantageously a disintegrating tablet and even more preferably a tablet that disperses in the mouth, a powder or a gelatin capsule, characterized in that it comprises microcapsules such as described above.

Furthermore, the invention relates to the use of microparticles such as defined above for the preparation of microparticulate oral galenical (pharmaceutical or dietetic) forms, preferably as tablets, advantageously tablets that disperse in the mouth, powders or gelatin capsules.

Finally, the invention further relates to a method of therapeutic treatment, characterized in that it consists in ingesting, according to a given dosage, a drug comprising microcapsules such as defined above.

The invention will be explained more clearly by the Examples below, given solely by way of illustration, which afford a good understanding of the invention and show its variants and/or modes of implementation, as well as its different advantages.

### EXAMPLES

### DESCRIPTION OF THE FIGURES

FIG. 1 shows the in vitro release profiles of the microcapsules of Example 1 at pH 1.4: —●—, and at pH 1.4 for 3 hours and then at pH 6.8 as from T=3 hours: —□—, in % by weight (% D) of dissolved metformin as a function of the time T in hours;

FIG. 2 shows the in vitro release profiles of the microcapsules of Example 2 at pH 1.4: —●—, and at pH 1.4 for 2 hours and then at pH 6.8 as from 2 hours: —□—, in % by weight (% D) of acyclovir as a function of the time T in hours;

FIG. 3 shows the in vitro release profiles of the microcapsules of Example 3 at pH 1.4: —▪—, and at pH 6.8: —●—, in % by weight (% D) of metformin as a function of the time T in hours.

### EXAMPLES

### Example 1

### Preparation of Microcapsules Allowing a Dual-mechanism Delayed and Prolonged Release of Metformin.HCl

75 g of metformin.HCl (Chemsource) and 75 g of PVP are dissolved in 1350 g of isopropanol. The solution is sprayed onto 850 g of neutral microspheres (NP Pharm) in a Glatte® GPCG3 spray coater.

93.3 g of hydrogenated palm oil (Hüls) (B) and 140 g of Eudragit® L100 (Röhm) (A) are dissolved in hot isopropanol. B/A=0.66. The solution is sprayed onto 700 g of previously prepared microparticles. The film coating conditions are: inlet temperature: 45° C., spraying rate: 8-12 g/min, atomization pressure: 1.5 bar.

The microcapsules were tested in a type II dissolutest according to the Pharmacopeia, at 37° C. and with agitation at 100 rpm, in the following media:
a) HCl at pH 1.4
b) HCl at pH 1.4 for 3 hours, then KH₂PO₄/NaOH buffer medium at pH 6.8

The release profiles are shown in FIG. 1.

These profiles are characteristic of a delayed and then prolonged release by means of a dual mechanism: absence of release for 2 hours, followed by a prolonged release without a change in pH, and finally followed by a release accelerated by the change in pH.

### Example 2

### Preparation of Microcapsules Allowing a Dual-mechanism Delayed and Prolonged Release of Acyclovir

75 g of acyclovir and 75 g of the polyvinylpyrrolidone PLASDONE® K29/32 are dissolved in 833 g of isopropanol. The solution is sprayed onto 850 g of neutral microspheres (NP Pharm) in a Glatt® GPCG3 spray coater.

93.3 g of hydrogenated palm oil (Hüls) (B) and 140 g of EUDRAGIT® L100 (Röhm) (A) are dissolved in hot isopropanol. B/A=0.66. The solution is sprayed onto 700 g of previously prepared microparticles. The film coating conditions are: inlet temperature: 45° C., spraying rate: 8-12 g/min, atomization pressure: 1.5 bar.

The microcapsules were tested in a type II dissolutest according to the Pharmacopeia, at 37° C. and With agitation at 100 rpm, in the following media:
c) HCl at pH 1.4
d) HCl at pH 1.4 for 3 hours, then KH₂PO₄/NaOH buffer medium at pH 6.8

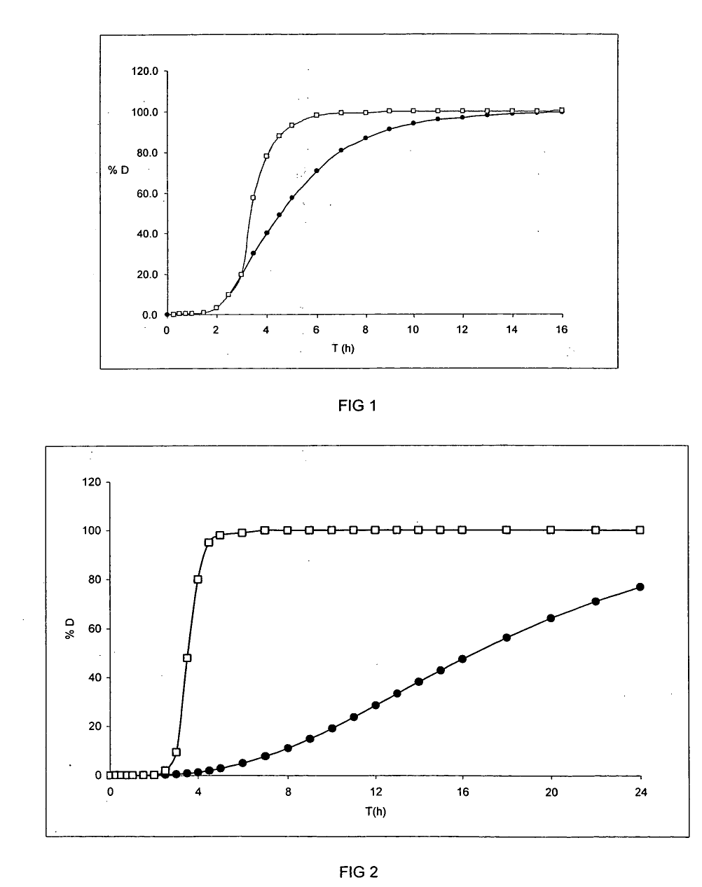

The release profiles are shown in FIG. 2.

The acyclovir release profile obtained at pH 1.4 is characteristic of a delayed and prolonged release by means of a dual release triggering mechanism.

### Example 3

### Preparation of Microcapsules Allowing a Dual-mechanism Delayed and Prolonged Release of Metformin.HCl

105 g of hydrogenated palm oil (Hüls) (B), 30 g of dibutyl sebacate and 165 g of Eudragit® L100 (Röhm) (A) are dissolved in hot isopropanol. B/A=0.64. The solution is sprayed onto 700 g of metformin granules (95.5% metformin/4.5% PVP). The film coating conditions are: inlet temperature: 45° C., spraying rate: 8-12 g/min, atomization pressure: 1.5 bar.

The microcapsules were tested in a type II dissolutest according to the Pharmacopeia, at 37° C. and with agitation at 100 rpm, in the following media:
e) HCl at pH 1.4
f) KH₂PO₄/NaOH buffer medium at pH 6.8

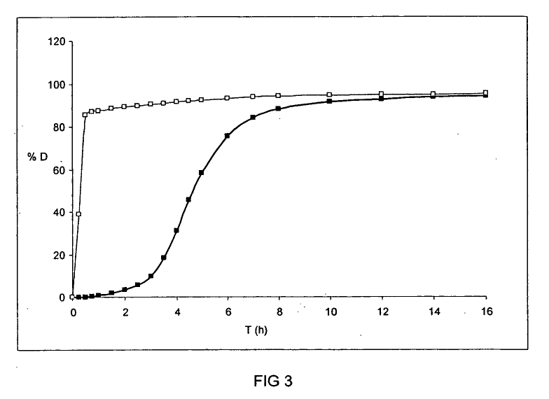

The release profiles are shown in FIG. 3.

These profiles are characteristic of a delayed and then prolonged release by means of a dual mechanism: absence of release for 2 hours at acidic pH and rapid release at neutral pH.

1. Microparticulate oral galenical form for the delayed and controlled release of at least one AP—excluding perindopril—this AP having an absorption window in vivo that is essentially limited to the upper parts of the gastrointestinal tract,
said form being designed so as to guarantee its therapeutic efficacy by guaranteeing its absorption in vivo, and being characterized in that:
the release of the AP is governed by two different triggering mechanisms, one being based on a variation in pH and the other allowing the release of the AP after a predetermined residence time in the stomach,
and its dissolution behavior in vitro (determined as indicated in the European Pharmacopeia, 3rd edition, under the title: “Dissolution test for solid oral forms”: type II dissolutest performed under SINK conditions, maintained at 37° C. and agitated at 100 rpm) is such that:
at a constant pH of 1.4, the dissolution profile includes a latency phase with a duration less than or equal to 5 hours, preferably of between 1 and 5 hours,
and the change from pH 1.4 to pH 6.8, during the latency phase, results in a release phase that starts without a latency period.

2. Galenical form according to claim 1, characterized in that it comprises “reservoir” microcapsules containing at least one active principle (AP)—excluding perindopril—these microcapsules being of the type that:
consist of particles of AP each coated with at least one film, this coating film consisting of a composite material which:
comprises:
at least one hydrophilic polymer A carrying groups that are ionized at neutral pH,
and at least one hydrophobic compound B;
and represents a mass fraction (% by weight, based on the total mass of the microcapsules) of ≦40;
and have a diameter below 2000 microns, preferably of between 200 and 800 microns and particularly preferably of between 200 and 600 microns,
characterized in that their coating film consists of a composite based on A and B in which:
the weight ratio B/A is between 0.2 and 1.5, preferably between 0.5 and 1.0,
and the hydrophobic compound B is selected from products that are crystalline in the solid state and have a melting point T_{fB} such that T_{fB}≧40° C., preferably T_{fB}≧50° C. and particularly preferably 40° C.≦T_{fB}≦90° C.

3. Galenical form according to claim 2, characterized in that the hydrophilic polymer A is selected from:
(meth)acrylic acid/alkyl (e.g. methyl) (meth)acrylate copolymers and mixtures thereof;
cellulose derivatives, preferably cellulose acetate and/or phthalate, hydroxypropyl methyl cellulose phthalate and hydroxypropyl methyl cellulose acetate and/or succinate;
and mixtures thereof.

4. Galenical form according to claim 2 or 3, characterized in that the compound B is selected from the following group of products:
vegetable waxes, taken on their own or in mixtures with one another;
hydrogenated vegetable oils, taken on their own or in a mixture with one another, preferably selected from the group comprising hydrogenated cottonseed oil, hydrogenated soybean oil, hydrogenated palm oil and any mixtures thereof;
monoesters and/or diesters and/or triesters of glycerol with at least one fatty acid, preferably behenic acid, taken by themselves or in a mixture with one another;
and mixtures thereof.

5. Galenical form according to claim 1, characterized in that, at a constant pH of 1.4, the controlled release phase following the latency phase is such that the release time for 50% of the AP (t_{1/2}) is defined as follows (in hours):
0.25≦t_{1/2}≦35
preferably 0.5≦t_{1/2}≦20

6. Galenical form according to claim 1, characterized in that the release phase following the change from pH 1.4 to pH 6.8, which takes place without a latency period, is such that the release time for 50% of the AP (t_{1/2}) is defined as follows (in hours):
0.25≦t_{1/2}≦20
preferably 0.5≦t_{1/2}≦15

7. Galenical form according to any one of claims 1 to 6, characterized in that the microcapsules comprise a single composite coating film AB.

8. Galenical form according to any one of claims 1 to 7, characterized in that the AP is deposited on a neutral core with a diameter of between 200 and 800 microns, preferably of between 200 and 600 microns.

9. Galenical form according to claim 8, characterized in that the neutral core contains sucrose and/or dextrose and/or lactose.

10. Galenical form according to claim 8, characterized in that the neutral core is a cellulose microsphere.

11. Galenical form according to any one of claims 1 to 10, characterized in that the AP used belongs to at least one of the following families of active substances: antiulcer agents, antidiabetics, anticoagulants, antithrombics, hypolipidemics, antiarrhythmics, vasodilators, antiangina agents, antihypertensives, vasoprotectors, fertility promoters, labor inducers and inhibitors, contraceptives, antibiotics, antifungals, antivirals, anticancer agents, antiinflammatories, analgesics, antiepileptics, antiparkinsonian agents, neuroleptics, hypnotics, anxiolytics, psychostimulants, antimigraine agents, antidepressants, antitussives, antihistamines and antiallergics.

12. Galenical form according to claim 11, characterized in that the AP is selected from the following compounds: amoxicillin, metformin, acetylsalicylic acid, pentoxifyllin, prazosin, acyclovir, nifedipine, diltiazem, naproxen, ibuprofen, flurbiprofen, ketoprofen, fenoprofen, indomethacin, diclofenac, fentiazac, estradiol valerate, metoprolol, sulpiride, captopril, cimetidine, zidovudine, nicardipine, terfenadine, atenolol, salbutamol, carbamazepine, ranitidine, enalapril, simvastatin, fluoxetine, alprazolam, famotidine, ganciclovir, famciclovir, spironolactone, 5-asa, quinidine, morphine, pentazocine, paracetamol, omeprazole, metoclopramide and mixtures thereof.

13. Galenical form according to any one of claims 1 to 12, characterized in that it is a tablet, advantageously a tablet that disperses in the mouth, a powder or a gelatin capsule.

14. Use of the microcapsules as defined in any one of claims 1 to 12 for the preparation of microparticulate oral galenical (pharmaceutical or dietetic) forms, preferably as tablets, advantageously tablets that disperse in the mouth, powders or gelatin capsules.
